SEQUENCE LISTING (10 sequences)

SEQ ID NO: 1 - DNA, 61 bp, Artificial Sequence
  Description of Artificial Sequence primer
aatgagcaaagtcagactcgcaattatcggtaacggtatggcgaataaatacctgtgacgg

SEQ ID NO: 2 - DNA, 63 bp, Artificial Sequence
  Description of Artificial Sequence primer
ggctcatgcgttgtcctccaccagagttactgggatacgtttacgccccgccctgccactcat

SEQ ID NO: 3 - DNA, 945 bp, Artificial Sequence
  Description of Artificial Sequence cat gene flanked by regions for integration
aatgagcaaagtcagactcgcaattatcggtaacggtatggcgaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgtatcccagtaactctggtggaggacaacgcatgagcc

SEQ ID NO: 4 - DNA, 24 bp, Artificial Sequence
  Description of Artificial Sequence primer
agccgtcaccgtcagcataacacc

SEQ ID NO: 5 - DNA, 24 bp, Artificial Sequence
  Description of Artificial Sequence primer
gcacgctggactcgaagaacgggt

SEQ ID NO: 6 - DNA, 5476 bp, Escherichia coli
gtgacttaagaaaatttatacaaatcagcaatatacccattaaggagtatataaaggtgaatttgatttacatcaataagcggggttgctgaatcgttaaggtaggcggtaatagaaaagaaatcgaggcaaaaatgagcaaagtcagactcgcaattatcggtaacggtatggtcggccatcgctttatcgaagatcttcttgataaatctgatgcggccaactttgatattaccgttttctgtgaagaaccgcgcatcgcttatgaccgcgtacacctctcgtcttacttctctcaccacaccgccgaagagctgtcgctggtgcgcgaaggcttctacgagaaacacggcatcaaagttctggtcggcgaacgcgctatcaccatcaaccgtcaggagaaggtgattcactccagcgccggacgtaccgttttttatgacaagctgatcatggcaaccggttcctacccgtggatcccgccaatcaaaggttctgatactcaggactgctttgtctatcgcactattgaagacctcaacgccattgaatcctgcgcccgtcgcagcaaacgcggtgccgttgttggtggcggcctgttaggtctggaagccgcaggcgcgctgaaaaacttaggtattgaaacccacgttatcgaatttgcccctatgctgatggcagaacagcttgatcagatgggcggcgagcagctgcgtcgcaaaatcgaaagtatgggcgtgcgcgttcacaccagcaaaaacacccttgagattgtgcaggaaggtgttgaagcgcgtaaaaccatgcgttttgccgacggcagcgaactggaagtcgactttatcgtcttctctaccggtatccgtccgcgcgataagctggcaacccagtgtggtctggacgttgctccgcgtgggggtattgtcattaatgattcctgccagacttccgatccggatatctacgccatcggtgaatgcgcaagctggaacaaccgtgtatttggtctggtagcacctggctacaaaatggcgcaggtcgccgttgaccatattctcggtagcgaaaacgcctttgaaggtgctgaccttagcgccaagctgaaactgctgggcgtagacgtaggcggtattggtgatgcgcacggtcgcacgcctggcgcacgtagctacgtttacctcgacgaaagtaaagagatctacaaacgcctgattgtcagcgaagacaacaaaaccctgctcggtgcggtactggtgggcgataccagcgactacggtaacctgctgcaactggtgctgaacgctatcgaactgccggaaaacccggattccctgatcctgccagcacactcgggtagcggcaagccgtctatcggtgttgataaactgccggacagcgcgcaaatctgctcctgcttcgacgtcaccaaaggtgatctgattgctgccatcaacaaaggctgccacacagttgcggcgctgaaagctgaaaccaaagcgggtactggctgcggtggctgtatcccgctggtcactcaggtactgaacgcggaactggcgaaacagggcatcgaagttaacaacaacctgtgcgaacactttgcttattcgcgtcaggaactgttccatttgatccgcgttgaaggcattaaaaccttcgaagaactgctggcgaaacacggcaaaggctacggttgtgaagtttgtaaaccaaccgtcggttcgctgctggcctcctgctggaacgaatacattctgaagccggaacatactccgctgcaggattctaacgacaacttcctcgctaacatccagaaagacggcacctactcggtgatcccgcgttctccgggcggtgaaatcaccccggaagggctgatggcggtaggtcgtatcgcgcgtgaatttaatctctacaccaagatcactggctcccagcgtctggcgatgtttggcgcacagaaagacgatctgccggagatctggcgtcagctgattgaagccggcttcgaaaccggtcatgcctatgcgaaagcactgcgtatggcgaaaacctgcgtgggtagcacctggtgccgctacggcgttggcgacagcgtcggcctcggcgtggaactggaaaaccgctacaaaggcatccgtacgccgcacaaaatgaagttcggtgtctccggctgtacccgtgaatgttcagaagctcagggtaaagacgtgggtattatcgccactgaaaaaggctggaacctgtatgtttgcggtaacggcggcatgaaaccgcgtcatgcggatctgctggcggcggatatcgatcgcgaaacgctgatcaaatatctcgaccgcttcatgatgttctacatccgtactgccgacaaactgacgcgtaccgcaccgtggttagaaaacctcgaaggcggcatcgattacctgaaagcagtgatcattgacgacaaactggggctgaacgcacatctggaagaagagatggcgcgcctgcgtgaagcggtactgtgtgagtggactgaaacggtcaatacgccgtctgcgcagactcgcttcaaacacttcatcaacagcgacaagcgtgacccgaacgtgcagatggtgccagagcgcgaacagcaccgtccggcaacgccgtatgaacgtatcccagtaactctggtggaggacaacgcatgagccagtggaaagacatctgcaaaatcgatgacatcctgcctgaaaccggcgtctgcgcgctgttaggtgacgagcaggtcgcgattttccgcccgtatcacagcgatcaggtgtttgcgatcagcaacatcgacccgttcttcgagtccagcgtgctgtcacgcggactgattgcggaacaccagggcgagctgtgggtcgccagcccgctgaaaaaacagcgttttcgcttaagcgacggcttgtgcatggaagacgaacagttttccgtcaaacattacgaagcgcgagtgaaagacggcgtggtgcagctgcgcggttaatgttttaacgggaggcgcaatgcctcccctttttgcatggtcctgtaataatcttcggtatattgcaggacattttttaaactttttgttttattttttgtttttattttttaaaggataatcaaatgtttacagacactattaataagtgtgcggctaacgctgcgcgcattgcacgcctgtcggcaaataacccgctcggcttttgggtcagctccgccatggcgggcgcgtatgtgggtcttgggatcatcctgattttcacgctcggtaatttgctcgatccatccgtacgccctttggtgatgggcgcgacctttggtatcgccttaacgctggtgattatcgccggttctgaactgttcaccggacacaccatgttcctcacctttggggtaaaagcgggcagcatcagccacgggcaaatgtgggcaatcctgccgcaaacctggctgggtaacctggtcggttccgtcttcgttgccatgctctatagctggggcggcggtagcctgctgccggtagataccagcatcgttcactccgtcgcgctggctaaaaccactgcaccggcaatggtactcttcttcaaaggtgcattgtgtaactggctggtttgcctggcaatctggatggcgctgcgcactgaaggggcggcgaaatttatcgctatctggtggtgtctgctggcatttatcgcgtccggctacgagcactctatcgctaacatgacgctgttcgcgctctcctggttcggcaaccacagcgaagcctacacgctggcgggtattggtcataacctgctgtgggtgacgctgggtaatactttatcaggtgccgtattcatgggattgggttattggtatgctacgccgaaagcgaatcgtccggttgcggacaaatttaatcaaactgaaacggctgccggttaattactaaggggtttttacgtggatcatttgcctatattttgccaattacgcgatcgcgactgtctgattgtcggcggtggtgatgtcgcggaacgcaaagcaaggttgctgttagacgcaggcgctcgcttaacggtgaatgcattagcgtttattccacagttcaccgcatgggcagatgcaggcatgttaaccctcgtcgaagggccatttgatgaaagccttctcgacacctgctggctggcgattgcagcgacggatgatgacgcgcttaaccagcgcgtcagcgaagccgctgaagctcgtcgcatcttctgtaacgtggtcgatgcgccgaaagccgccagctttattatgccgtcgattattgaccgctcaccgctcatggtagcggtctcctctggcggcacctctccggttctggcacgcctgttgcgcgaaaaacttgaatcactgctgccgttacatctgggccaggtagcgaaatacgccgggcaattacgcgggcgagtgaaacaacagttcgccacgatgggtgagcgtcgccgtttctgggagaaattgttcgttaacgaccgcctggcgcagtcgctggcaaacaacgatcagaaagccattactgaaacgaccgaacagttaatcaacgaaccgctcgaccatcgcggtgaagtggtgctggttggtgcaggtccgggcgatgccgggctgctgacactgaaaggactgcaacaaattcagcaggcagatgtggtggtctacgaccgtctggtttctgacgatattatgaatctggtacgccgcgatgcggaccgtgttttcgtcggcaaacgcgcgggataccactgcgtaccccaggaagagattaaccagatcctgctgcgggaagcgcaaaaaggcaaacgcgtggtgcggctgaaaggtggcgatccgtttatttttggccgtggtggcgaagagctggaaacactgtgcaacgcgggtattccgttctcggtggttccgggtattaccgcagcttctggttgctctgcctattcgggtattccactcacgcatcgcgattatgcccagagcgtacgcttaattaccggacacttaaaaaccggtggcgagctggactgggaaaacctggcggcagaaaaacagacgctggtgttctatatggggttgaatcaggccgcgactattcagcaaaagctgattgaacacggaatgccaggcgaaatgccggtggcaattgtcgaaaacggtacggcagtcacgcagcgcgtgattgacggtacgctcacacagctgggagaactggcgcagcaaatgaacagtccatcgctaattattattggtcgggttgttggcctgcgcgataaactgaactggttctccaaccattaatttaacccgggccagagaattctggccttcttaatgactctttttatatattcagcaaataaaacatagccccttaataaatattatctgctaatgggttatattcaccgttacagcgacttacaaaattaagtcattcgctcactgacgc

SEQ ID NO: 7 - protein, 847 aa, Escherichia coli
MSKVRLAIIGNGMVGHRFIEDLLDKSDAANFDITVFCEEPRIAYDRVHLSSYFSHHTAEELSLVREGFYEKHGIKVLVGERAITINRQEKVIHSSAGRTVFYDKLIMATGSYPWIPPIKGSDTQDCFVYRTIEDLNAIESCARRSKRGAVVGGGLLGLEAAGALKNLGIETHVIEFAPMLMAEQLDQMGGEQLRRKIESMGVRVHTSKNTLEIVQEGVEARKTMRFADGSELEVDFIVFSTGIRPRDKLATQCGLDVAPRGGIVINDSCQTSDPDIYAIGECASWNNRVFGLVAPGYKMAQVAVDHILGSENAFEGADLSAKLKLLGVDVGGIGDAHGRTPGARSYVYLDESKEIYKRLIVSEDNKTLLGAVLVGDTSDYGNLLQLVLNAIELPENPDSLILPAHSGSGKPSIGVDKLPDSAQICSCFDVTKGDLIAAINKGCHTVAALKAETKAGTGCGGCIPLVTQVLNAELAKQGIEVNNNLCEHFAYSRQELFHLIRVEGIKTFEELLAKHGKGYGCEVCKPTVGSLLASCWNEYILKPEHTPLQDSNDNFLANIQKDGTYSVIPRSPGGEITPEGLMAVGRIAREFNLYTKITGSQRLAMFGAQKDDLPEIWRQLIEAGFETGHAYAKALRMAKTCVGSTWCRYGVGDSVGLGVELENRYKGIRTPHKMKFGVSGCTRECSEAQGKDVGIIATEKGWNLYVCGNGGMKPRHADLLAADIDRETLIKYLDRFMMFYIRTADKLTRTAPWLENLEGGIDYLKAVIIDDKLGLNAHLEEEMARLREAVLCEWTETVNTPSAQTRFKHFINSDKRDPNVQMVPEREQHRPATPYERIPVTLVEDNA

SEQ ID NO: 8 - protein, 108 aa, Escherichia coli
MSQWKDICKIDDILPETGVCALLGDEQVAIFRPYHSDQVFAISNIDPFFESSVLSRGLIAEHQGELWVASPLKKQRFRLSDGLCMEDEQFSVKHYEARVKDGVVQLRG

SEQ ID NO: 9 - protein, 184 aa, Escherichia coli
MFLTFGVKAGSISHGQMWAILPQTWLGNLVGSVFVAMLYSWGGGSLLPVDTSIVHSVALAKTTAPAMVLFFKGALCNWLVCLAIWMALRTEGAAKFIAIWWCLLAFIASGYEHSIANMTLFALSWFGNHSEAYTLAGIGHNLLWVTLGNTLSGAVFMGLGYWYATPKANRPVADKFNQTETAAG

SEQ ID NO: 10 - protein, 457 aa, Escherichia coli
VDHLPIFCQLRDRDCLIVGGGDVAERKARLLLDAGARLTVNALAFIPQFTAWADAGMLTLVEGPFDESLLDTCWLAIAATDDDALNQRVSEAAEARRIFCNVVDAPKAASFIMPSIIDRSPLMVAVSSGGTSPVLARLLREKLESLLPLHLGQVAKYAGQLRGRVKQQFATMGERRRFWEKLFVNDRLAQSLANNDQKAITETTEQLINEPLDHRGEVVLVGAGPGDAGLLTLKGLQQIQQADVVVYDRLVSDDIMNLVRRDADRVFVGKRAGYHCVPQEEINQILLREAQKGKRVVRLKGGDPFIFGRGGEELETLCNAGIPFSVVPGITAASGCSAYSGIPLTHRDYAQSVRLITGHLKTGGELDWENLAAEKQTLVFYMGLNQAATIQQKLIEHGMPGEMPVAIVENGTAVTQRVIDGTLTQLGELAQQMNSPSLIIIGRVVGLRDKLNWFSNH

(19) United States
(12) Patent Application Publication
(10) Pub. No.: US 2005/0069994 A1
(43) Pub. Date: Mar. 31, 2005

(54) Method for producing L-amino acid using bacterium of Enterobacteriaceae family, having nir operon inactivated

(76) Inventors: Leonid Romanovich Ptitsyn, Moscow (RU); Irina Borisovna Altman, Moscow (RU); Sergey Vasil'evich Smirnov, Moscow (RU); Natalia Nikolaevna Samsonova, Moscow (RU); Vladimir Yurievich Ermishev, Moscow (RU)

Correspondence Address: AJINOMOTO CORPORATE SERVICES, LLC;INTELLECTUAL PROPERTY DEPARTMENT, 1120 CONNECTICUT AVE., N.W., WASHINGTON, DC, 20036, US

(21) Appl. No.: 10/927,040

(22) Filed: Aug. 27, 2004

(30) Foreign Application Priority Data
Aug. 29, 2003 (RU) 2003126289

(51) Int. Cl. C12P 13/10; C12P 21/04; C12N 1/21

(52) U.S. Cl. 435/114; 435/252.33

(57) ABSTRACT

A method is provided for producing L-amino acid, such as L-arginine using a bacterium of Enterobacteriaceae family, particularly a bacterium belonging the genus Escherichia, with an inactivated nir operon.

### BACKGROUND OF THE INVENTION

1. Field of the Invention

The present invention relates to the microbiological industry, and specifically to a method for producing an L-amino acid using bacterium of Enterobacteriaceae family, wherein the nir operon, including the nirBDC-cysG genes, is inactivated.

2. Description of the Related Art

Escherichia coli possesses two biochemically distinct nitrite reductase enzymes encoded by the nrfABCDEFG and nirBDC operons, respectively (Cole, J., FEMS Microbiol. Lett. 136: 1-11 (1996)). A basal expression level of the nir operon is about 8 times higher than that of the nrf operon and can be increased 21 -fold by adding nitrate (Wang, H. and Gunsalus, H. P., J. Bacteriol., 182, No. 20, p. 5813-5822 (2000)). Transcription of the nirBDC operon is driven from a single promoter, and expression is activated by two environmental signals: an absence of oxygen and a presence of nitrite or nitrate ions in the growth medium (Jayaraman et al., J. Mol. Biol., 196, 4:781-8 (1987); Page et al., Arch Microbiol., 154:4:349-54, (1990)). Also, the cysG gene is co-transcribed with the nirBDC operon, while the second constitutive promoter is located less than 100 bp upstream of the cysG gene (Peakman, T. et al, Eur. J. Biochem., 191(2):325-331 (1990)).

The product of the cysG gene, siroheme synthase, catalyzes the synthesis of a heme cofactor, siroheme, which is employed by sulfite reductase and nitrite reductase enzymes in the sulfate and nitrite reduction processes.

The NirBDC nitrite reductase is a siroheme-containing enzyme that uses NADH as an electron donor to reduce nitrite in the cytoplasm (MacDonald, H. and Cole, J., Mol. Gen. Genet., 200:320-334 (1985); Peakman, T. et al, Eur. J. Biochem., 191:315-323 (1990)). E. coli mutants defective in the nirB gene lack NADH-dependent nitrite reductase activity and reduce nitrite slowly during anaerobic growth. These mutants require cysteine for growth (Cole, J. A. et al, J. Gen. Microbiol. 120:475-483 (1980)).

The auxotrophy of cysG mutants is a result of their failure to produce siroheme, the cofactor of the CysIJ enzyme, sulfite reductase. Siroheme-dependent sulfite reduction is required for synthesis of cysteine, methionine, and other sulfur-containing metabolites whenever sulfate or sulfite is utilized as a sulfur source in the synthesis of these metabolites (Becker, M. A. et al, J. Biol. Chem., 244:2418-2427 (1969); Becker, M.A. and Tomkins, G. M., J. Biol. Chem., 244:6023-6030 (1969)). Since siroheme is also required for nitrite reductase (NirB), cysG mutants are also defective in reduction of nitrite. The nir promoter (Pₙᵢᵣ) contains an FNR binding site (position −41.5); a NarL/NarP binding site (position −69.5) (Jayaraman, P. S. et al, Nucleic Acids Res. 17:1 135-45 (1989); Tyson, K. L. et al, Mol. Microbiol., 7:1:151-7 (1993)); a Fis binding sites (−142, +23); an IHF binding site (−88); and a binding site for the nucleoid associated protein, H-NS, which preferentially binds to upstream sequences at the nir promoter.

The nir promoter is repressed by the following three DNA binding proteins: Fis, IHF and H-NS. The activation of nir promoter expression is co-dependent on both the FNR protein (an anaerobically triggered transcription activator) and the NarL or NarP proteins (transcription activators triggered by nitrite and nitrate). Under anaerobic conditions, FNR binds to a site −41.5, activating transcription of the nir operon. The nir promoter is further regulated by the presence of nitrite or nitrate ions in the medium. This is achieved by two very similar response-regulator family transcription factors, NarL and NarP (reviewed by Darwin, A. J. et al, Mol. Microbiol., 20:3:621-32 (1996)). In response to nitrite or nitrate, NarL and NarP are phosphorylated by the membrane-bound sensor kinase proteins, NarX and NarQ. Phosphorylated NarL and NarP then bind to specific heptamer sequences at target promoters and either up- or down-regulate transcription initiation at these promoters (for examples, see Tyson, K. L. et al., Mol. Microbiol., 13:6:1045-55 (1994); Darwin, A. J. et al., Mol. Microbiol., 25:3:583-95 (1997)).

The association of Fis, IHF and H-NS suggests that nir promoter DNA is sequestrated into a highly ordered nucleo-protein structure that represses FNR-dependent transcription activation. NarL and NarP can relieve both IHF- and Fis-mediated repression, but are unable to counteract H-NS mediated repression (Browning D. F. et al, Molecular Microbiology, 37(5), 1258-1269 (2000)).

The high nitrite conditions needed for nirB induction are consistent with the proposed role of the NirB enzyme in detoxification (Fazzio, T. G., and Roth, J. R., J. Bacteriol. 178:6952-6959 (1996)). A second plausible role for the NirB enzyme is thatit recycles NADH by oxidizing it in the presence of excess reducing equivalents. Such conditions occur when sufficient energy is generated by nitrate-dependent respiration via the NarG nitrate reductase complex. The presence of the NirB enzyme would thus allow the cell to effectively decouple carbon dissimilation from the nitrite respiratory pathways by using a futile cycle for NADH-AND recycling (Wang, H. and Gunsalus, R. P., J. Bacteriol., 182, No. 20, p. 5813-5822 (2000)).

There have been no reports to date describing inactivation of the nir operon for the purpose of producing L-amino acids.

### SUMMARY OF THE INVENTION

An object of present invention is to enhance the productivity of L-amino acid producing strains. It is a further object of the invention to provide a method for producing L-amino acids using these strains.

It is a further object of the present invention to provide an L-amino acid- producing bacterium of the Enterobacteriaceae family, wherein the bacterium has been modified to inactivate the nir operon.

It is a further object of the present invention to provide the L-amino acid-producing bacterium as described above, wherein said nir operon comprises the nirBDC and cysG genes.

It is a further object of the present invention to provide the bacterium as described above, wherein the bacterium belongs to the genus Escherichia.

It is a further object of the present invention to provide the L-amino acid producing bacterium as described above, wherein said L-amino acid is L-arginine.

It is a further object of the present invention to provide the L-amino acid-producing bacterium as described above, wherein the bacterium has been modified to enhance expression of an L-arginine operon.

It is a further object of the present invention to provide a method for producing L-amino acid comprising:
cultivating the bacterium as described above in a medium, and
collecting the accumulated L-amino acid from the medium.

It is a further object of the present invention to provide the method as described above, wherein said L-amino acid is L-arginine.

It is a further object of the present invention to provide the method as described above, wherein the bacterium has been modified to enhance expression of an L-arginine operon.

### BRIEF DESCRIPTION OF DRAWINGS

FIG. 1 shows the relative positions of the primers nirBL and nirBR on the plasmid pACYC184, which is used for amplification of cat gene.

FIG. 2 shows the construction of the chromosomal DNA fragment, which contains an inactivated nir operon.

### DETAILED DESCRIPTION OF THE INVENTION

The aforementioned objects were achieved by finding that the inactivation of the nir operon can enhance production of L-amino acids such as L-arginine. Thus, the present invention has been completed.

The present invention is described in details below.

Bacterium of the Present Invention

The bacterium of the present invention is an L-amino acid-producing bacterium of Enterobacteriaceae family, wherein the bacterium has been modified to inactivate the nir operon.

In the present invention, “L-amino acid-producing bacterium” means a bacterium, which has an ability to produce and cause accumulation of an L-amino acid in a medium, when the bacterium of the present invention is cultured in the medium. The L-amino acid-producing ability may be imparted or enhanced by breeding. The term “L-amino acid-producing bacterium” as used herein may also mean a bacterium which is able to produce and cause accumulation of L-amino acid in a culture medium in an amount larger than a wild-type or parental strain of bacterium, such as E. coli K-12 strain.

The bacterium of Enterobacteriaceae family that can be used in the present invention is not particularly limited, however, for example, bacteria described by Neidhardt, F. C. et al. (Escherichia coli and Salmonella typhimurium, American Society for Microbiology, Washington D.C., 1208, Table 1) are encompassed. Specifically, the Enterobacteriaceae family of bacteria includes bacteria belonging to the genera Escherichia, Erwinia, Providencia and Serratia. The genus Escherichia is preferred.

The phrase “a bacterium belonging to the genus Escherichia” means that the bacterium is classified as the genus Escherichia according to the classification known to a person skilled in the art of microbiology. A microorganism belonging to the genus Escherichia as used in the present invention inludes, but is not limited to, Escherichia coli (E. coli), which is most preferred bacterium for the present invention.

The phrase “nir operon is inactivated” or “to inactivate the nir operon” means that the target operon is modified in such a way that the modified gene of the operon encodes a mutant protein with decreased or no activity. It is also possible that the modified DNA region is unable to naturally express the operon due to the deletion of a part of the operon, shifting the reading frame of the operon gene(s), or the modification of adjacent regions of the operon, including sequences which control operon expression, such as promoter(s), enhancer(s), attenuator(s) etc.. The expression of the nir operon is driven from a single promoter located upstream of the nirB gene, and the constitutive basal expression level of the cysG gene is not sufficient for siroheme synthesis, due to its own weak promoter., It is possible, therefore, to inactivate only the nirB gene so that further expression of genes located downstream of the nirB gene becomes impossible. The role of nitrite reductase, encoded by the nirB gene, in L-arginine production remains unclear. One possible explanation is that inactivation of the nirB gene blocks transcription of the cysG gene from the nirB promoter and expression of the cysG gene from its own weak promoter is not sufficient for siroheme synthesis. This leads to a deficiency in the synthesis of cysteine, methionine and other sulfur-containing metabolites inducing the L-arginine biosynthetic pathway. So, one embodiment of the present invention includes inactivation or disruption of the cysG gene.

The nir operon of E. coli includes the following consecutively located genes: nirB, nirD, nirC and cysG. The nirB and nirD genes encode a nitrite reductase. The nirC gene encodes a nitrite transporter. The cysG gene encodes a siroheme synthase. The nirB gene (gi:16131244; numbers 3491648 to 3494191 in the GenBank accession number NC_{—}000913.1), nirD gene (gi:16131245; numbers 3494188 to 3494514 in the GenBank accession number NC_{—}000913.1), nirC gene (gi:16132233; numbers 3494640 to 3495446 in the GenBank accession number NC_{—}000913.1) and cysG gene (gi:16131246; numbers 3495465 to 3496838 in the GenBank accession number NC_{—}000913.1) are located between the yhfC and yhfL ORFs on the E. coli strain K-12 chromosome. The nucleotide sequence of the nir operon from E. coli MG1655 strain is registered in GenBank under accession No. AE000412 U00096. The nucleotide sequence comprising the nirB, nirD, nirC and cysG genes of the MG1655 strain is shown in SEQ ID NO: 6. The amino acid sequences encoded by the nirB, nirD, nirC and cysG genes are shown in SEQ ID NOS: 7, 8, 9 and 10, respectively. The coding regions of the nirB, nirD, nirC and cysG genes in the nucleotide sequence of SEQ ID NO: 6 are 135-2678, 2675-3001, 3379-3933 and 3952-5325, respectively.

Inactivation of the gene can be performed by conventional methods, such as mutagenesis treatment using UV irradiation or nitrosoguanidine (N-methyl-N′-nitro-N-nitrosoguanidine) treatment, site-directed mutagenesis, gene disruption using homologous recombination or/and insertion-deletion mutagenesis (Datsenko K. A. and Wanner B. L., Proc. Natl. Acad. Sci. USA, 2000, 97:12: 6640-45) which is also called “Red-driven integration”.

The nir operon of a bacterium of Enterobacteriaceae family other than E. coli can also be inactivated by homologous recombination using the nir operon fragment from E. coli or a fragment of an inherent nir operon which may be a homologue to the E. coli nir operon. Such a nir operon homologue may have homology of not less than 70%, preferably not less than 80%, more preferably not less than 90%, and most preferably not less than 95% to the E. coli nir operon with respect to the nucleotide sequence of respective coding regions.

L-arginine Producing Bacterium.

As a parent strain which is to be modified to inactivate the nir operon, L-arginine-producing bacteria are encompassed.

Bacteria belonging to the genus Escherichia which produce L-arginine include, but are not limited to, E. coli strain 237 (VKPM B-7925) and it's derivative strains harboring a mutant N-acetylglutamate synthase (Russian Patent No. 2215783), and an arginine-producing strain into which the argA gene encoding N-acetylglutamate synthetase is introduced (Japanese Laid-Open Publication No. 57-5693), and the like. The strain 237 is a mutant resistant to a pyrimidine analog, 6-azauracil, which was derived from E. coli K12 ilvA::Tn5 using N-methyl-N′-nitro-N-nitrosoguanidine (NTG). The strain 237 was deposited at Russian National Collection of Industrial Microorganisms (VKPM) on Apr. 10, 2000, and received an accession number of VKPM B-7925, and was converted to an international deposit under the provisions of Budapest Treaty on May 18, 2001.

The bacterium of the present invention can be obtained by inactivation of nir operon in a bacterium which inherently has the ability to produce an L- amino acid. Alternatively, the bacterium of present invention can be obtained by imparting the ability to produce an L- amino acid to a bacterium which already has an inactivated nir operon.

Methods for preparation of plasmid DNA, digestion and ligation of DNA, transformation, selection of an oligonucleotide as a primer and the like may be ordinary methods well known to one skilled in the art. These methods are described, for instance, in Sambrook, J., Fritsch, E. F., and Maniatis, T., “Molecular Cloning A Laboratory Manual, Second Edition”, Cold Spring Harbor Laboratory Press (1989).

2. Method of the Present Invention

The method of the present invention is a method for producing an L-amino acid having the following steps: cultivating the bacterium of the present invention in a culture medium which results in production accumulation of the L-amino acid in the medium, and collecting the accumulated L-amino acid from the medium. More specifically, the method of the present invention is a method for producing L-arginine, which method includes the steps of cultivating the bacterium of the present invention in a culture medium,, and collecting the accumulated L-arginine from the medium.

In the present invention, the cultivation, collection and purification of L-amino acid from the medium and the like may be performed by conventional fermentation methods typically used for production of an amino acid from a bacterium.

A medium used for the culture may be either synthetic or natural, so long as the medium includes a carbon source and a nitrogen source and minerals and, if necessary, appropriate amounts of nutrients which the bacterium requires for growth.

The carbon source may include various carbohydrates such as glucose and sucrose, and various organic acids. Depending on the mode of assimilation of the used microorganism, alcohol including ethanol and glycerol may be used.

As the nitrogen source, various ammonium salts such as ammonia and ammonium sulfate, other nitrogen compounds such as amines, a natural nitrogen source such as peptone, soybean-hydrolysate, and digested fermentative microorganism may be used.

As minerals, potassium monophosphate, magnesium sulfate, sodium chloride, ferrous sulfate, manganese sulfate, calcium chloride, and the like may be used. As vitamins, thiamine, yeast extract and the like may be used.

The cultivation is preferably performed under aerobic conditions such as a shaking culture, and stirring culture with aeration, at a temperature of 20 to 40° C., preferably 30 to 38° C. The pH of the culture is usually between 5 and 9, preferably between 6.5 and 7.2. The pH of the culture can be adjusted with ammonia, calcium carbonate, various acids, various bases, and buffers. Usually, a 1 to 5-day cultivation leads to the accumulation of the target L-amino acid in the liquid medium.

After cultivation, solids such as cells can be removed from the liquid medium by centrifugation or membrane filtration, and then the L-amino acid can be collected and purified by ion-exchange, concentration and/or crystallization methods.

### EXAMPLES

The present invention will be more concretely explained with reference to the following non-limiting Examples. In the Examples, arginine is of L-configuration.

### Example 1

### Construction the Strain having an Inactivated nir operon

Deletion of the nirB Gene

Deletion of the nirB gene was performed by the method first developed by Datsenko and Wanner (Proc. Natl. Acad. Sci. USA, 2000, 97(12), 6640-6645) and called “Red-driven integration”. According to this procedure, the PCR primers nirBL (SEQ ID NO: 1) and nirBR (SEQ ID NO: 2), which are homologous to both regions adjacent to the nirB gene, and a gene conferring antibiotic resistance in the template plasmid were constructed. Plasmid pACYC184 (NBL Gene Sciences Ltd., UK) (GenBank/EMBL accession number X06403) was used as a template in PCR reaction. PCR was conducted as follows: denaturation step for 3 min at 95° C.; profile for two first cycles: 1 min at 95° C., 30 sec at 50° C., 40 sec at 72 ° C.; profile for the last 25 cycles: 30 sec at 95° C., 30 sec at 54° C., 40 sec at 72° C; final step: 5 min at 72° C.

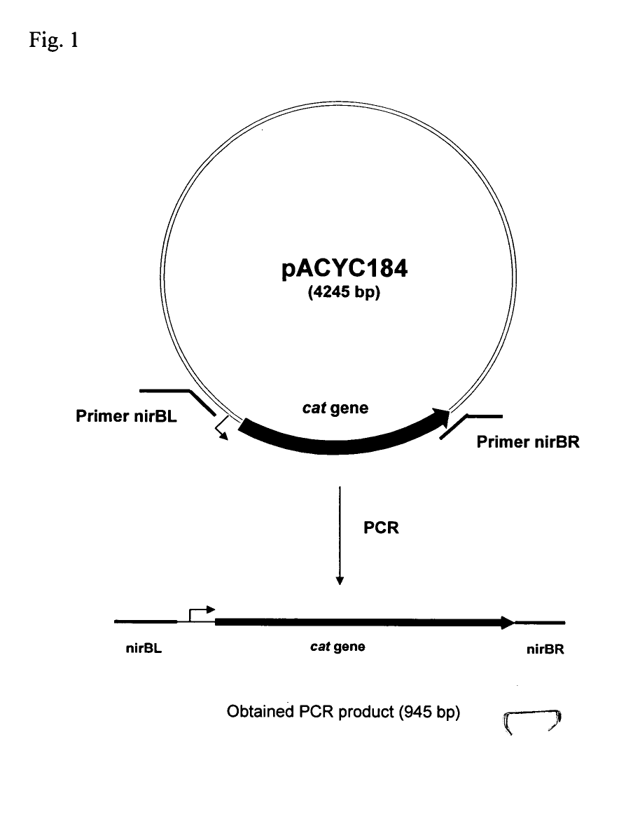

The obtained 945 bp PCR product (FIG. 1, SEQ ID NO: 3) was purified by agarose gel electrophoresis and used for electroporation of the E. coli strain MG1655 which harbors the plasmid pKD46 with temperature sensitive replication. The plasmid pKD46 (Datsenko and Wanner, Proc. Natl. Acad. Sci. USA, 2000, 97:12:6640-45) includes a 2,154 nucleotide (31088-33241) DNA fragment of phage k (GenBank accession No. J02459), which contains the λ Red homologous recombination system genes (γ, β, exo genes) under the control of the arabinose-inducible P_{araB} promoter. The plasmid pKD46 is necessary for integration of the PCR product into MG1655 strain chromosome.

Electrocompetent cells were prepared as follows: overnight culture of E. coli strain MG1655 grown at 30° C. in LB medium supplemented with ampicillin (100 mg/l) was diluted 100 times with 5 ml of SOB medium (Sambrook et al, “Molecular Cloning A Laboratory Manual, Second Edition”, Cold Spring Harbor Laboratory Press (1989)) with ampicillin and L-arabinose (1 mM). The obtained culture was grown with aeration at 30° C. to an OD₆₀₀ of ≈0.6 and then made electrocompetent by concentrating 100-fold and washing three times with ice-cold deionized H₂O. Electroporation was performed using 70 μl of a cell suspension and ≈100 ng of PCR product. After electroporation, cells were incubated with 1 ml of SOC medium (Sambrook et al, “Molecular Cloning A Laboratory Manual, Second Edition”, Cold Spring Harbor Laboratory Press (1989)) at 37° C. for 2.5 h and then plated onto an L-agar medium containing 25 mg/l of chloramphenicol (Cm) and grown at 37° C. to select Cm^{R} recombinants. Then, to eliminate the pKD46 plasmid, 2 passages on L-agar medium with Cm at 42° C. were performed and the obtained colonies were tested for sensitivity to ampicillin.

2. Verification of nirB Gene Deletion by PCR.

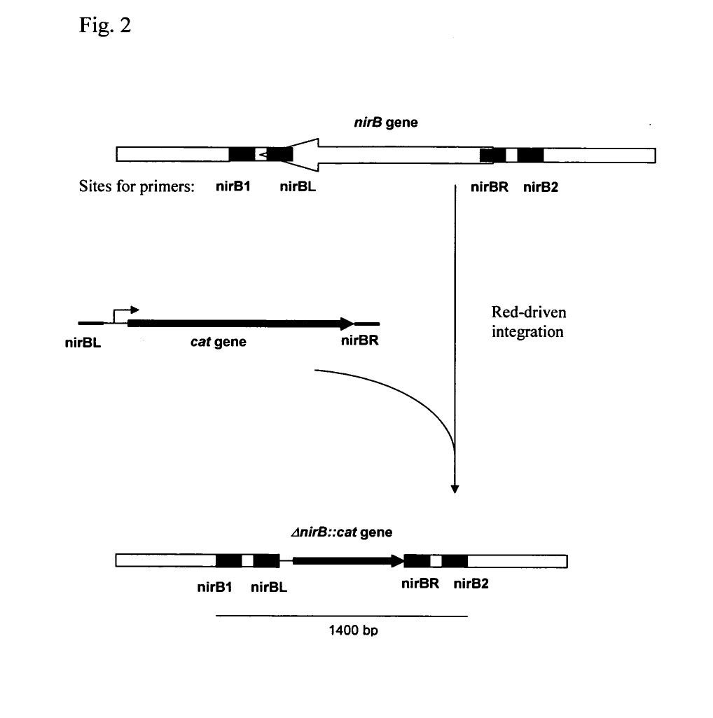

The mutants with the nirB gene deleted, marked with Cm resistance gene (cat), were verified by PCR. Locus-specific primers nirB1 (SEQ ID NO: 4) and nirB2 (SEQ ID NO: 5) were used for verification by PCR. Conditions for PCR verification were as follows: denaturation step for 3 min at 94° C.; profile for the 30 cycles: 30 sec at 94° C., 30 sec at 54° C., 1 min at 72° C.; final step: 7 min at 72° C. PCR product, obtained in the reaction with the cells of parental nirB⁺ strain MG1655 as a template, was 949 bp in length. The PCR product, obtained in the reaction with the cells of mutant MG1655 ΔnirB::cat strain as a template, was 1400 nucleotides in length (FIG. 2).

3. Construction of Arginine-Producing Strain with Inactivated nirB Gene.

The arginine-producing strain E. coli 237 (VKPM B-7925) was transduced to Cm resistance by the standard P1 transduction procedure (Sambrook et al, “Molecular Cloning A Laboratory Manual, Second Edition”, Cold Spring Harbor Laboratory Press (1989)). The strain MG1655 ΔnirB::cat was used as a donor for cat gene. The resulting strain 237ΔnirB::cat was verified by PCR to have ΔnirB::cat deletion by means of primers nirBI (SEQ ID NO: 4) and nirB2 (SEQ ID NO: 5).

### Example 2

### Production of L-arginine by E. coli Strain with Inactivated nirB Gene

Both E. coli strains 237 and 237AnirB::cat were grown overnight at 37° C. on L-agar plates. The strain 237ΔnirB::cat plate also contained chloramphenicol (20 μg/ml) . Then one loop of the cells was transferred to 2 ml of minimal medium for fermentation in the 20×200 mm test tubes. Cells were grown for 72 hours at 32° C. with shaking at 250 rpm.

After the cultivation, the amount of arginine which accumulated in the medium was determined by paper chromatography using arginine (1 g/l and 2 g/l) and glutamic acid (1 g/l and 2 g/l) as controls. The paper was developed with a mobile phase: n-butanol:acetic acid:water=4:1:1 (v/v). A solution of ninhydrin (0.5%) in acetone was used as a visualizing reagent.

The results are presented in Table 1.

The composition of the fermentation medium (g/l):

| | |
|---|---|
| Glucose | 67.0 |
| Yeast extract | 5.0 |
| (NH₄)₂SO₄ | 35.0 |
| KH₂PO₄ | 2.0 |
| MgSO₄.7H₂O | 2.0 |
| Thiamine (Vitamin B₁) | 1.0 |
| CaCO₃ | 25.0 |
| L-isoleucine | 0.05 |

Glucose and magnesium sulfate are sterilized separately. pH is adjusted to 7.2.

TABLE 1

| E. coli strain | OD₅₅₅ | Arg (g/l) | Glu(g/l) |
|---|---|---|---|
| 237 | 21.1 | 5.6 ± 0.5 | 4.6 ± 0.5 |
| 237ΔnirB::cat | 19.9 | 6.8 ± 0.6 | traces |

As it is seen from Table 1, inactivation of the nir operon improved the L-arginine accumulation by the L-arginine-producing strain 237.

While the invention has been described in detail with reference to preferred embodiments thereof, it will be apparent to one skilled in the art that various changes can be made, and equivalents employed, without departing from the scope of the invention. Each of the aforementioned documents, including the foreign priority document, RU 2003126289, is incorporated by reference herein in its entirety.

1. An L-amino acid-producing bacterium of the Enterobacteriaceae family, wherein the bacterium has been modified to inactivate the nir operon.

2. The L-amino acid-producing bacterium according to claim 1, wherein said nir operon comprises nirBDC and cysG genes.

3. The L-amino acid-producing bacterium according to claim 2, wherein said bacterium belongs to the genus Escherichia.

4. The L-amino acid-producing bacterium according to claim 1, wherein said L-amino acid is L-arginine.

5. The L-amino acid-producing bacterium according to claim 4, wherein said bacterium has been modified to enhance expression of an L-arginine operon.

6. A method for producing an L-amino acid comprising:
cultivating the bacterium according to claim 1 in a medium, and
collecting the accumulated L-amino acid from the medium.

7. The method according to claim 6, wherein said L-amino acid is L-arginine.

8. The method according to claim 7, wherein said bacterium has been modified to enhance expression of an L-arginine operon.
